(19) United States
(12) Patent Application Publication
(10) Pub. No.: US 2018/0353950 A1
(43) Pub. Date: Dec. 13, 2018

(54) Separator for Spectrophotometric Analysis of Body Fluids

(71) Applicant: Lightintegra Technology Inc., Vancouver (CA)

(72) Inventor: Elisabeth Maurer, Vancouver (CA)

(73) Assignee: Lightintegra Technology Inc., Vancouver (CA)

(21) Appl. No.: 15/997,743

(22) Filed: Jun. 5, 2018

Related U.S. Application Data
(60) Provisional application No. 62/519,077, filed on Jun. 13, 2017.

(51) Int. Cl. B01L 3/00; G01N 15/14; G01N 33/49

(52) U.S. Cl. CPC B01L 3/5027; G01N 15/1404; G01N 33/491; B01L 2200/0647; B01L 2400/0688; B01L 2400/0403; B01L 3/545

(57) ABSTRACT

An apparatus for separating components of a body fluid, especially whole blood, to prepare a sample that may be analyzed using spectrophotometric techniques such as dynamic light scattering (DLS) to assess the composition of the sample are disclosed. A microfluidic separator may be defined by a capillary tube having red blood cell traps incorporated therein; importantly, the microfluidic separator does not activate platelets as the whole blood flows through the separator by air replacement action (i.e., suction). Whole blood is processed to remove red blood cells so that DLS may be used to analyze the separated sample to detect merosomes in the sample. A method for diagnosing a pathological condition in a patient based on a body fluid from the patient comprises using a DLS instrument to collect DLS measurements from the body fluid; using the DLS measurements to detect a presence of merosomes in the body fluid; and diagnosing the pathological condition based on the presence of the merosomes, the presence of the detected merosomes being indicative of the existence of the pathological condition in the patient.

### TECHNICAL FIELD

This application relates in general to apparatus for use in testing body fluids such as blood, and more specifically to microfluidic separator apparatus for separating blood components for analysis using spectrophotometry and, more particularly, to spectrophotometric analysis for the detection of merosomes.

### BACKGROUND OF THE INVENTION

Analysis of “merosomes,” also known as and referred to as “microparticles,” in body fluids can be important and useful for diagnosing pathological conditions. The word “merosome” means body part; these are literally parts of other human body cells and as such they can take on activities of parent cells. In the case of platelets it has been shown that platelet-derived merosomes have much higher prothrombotic potency than platelets themselves—research is ongoing to find out why this might be. There is a large body of literature emerging that merosomes from platelets are beneficial to treat bleeding. Thus, merosome detection and analysis should focus on optimizing the use of donors and products because what might not be good for one patient might be optimal for another.

As an example of the potential benefits derived from analysis of merosomes, many normal blood donors have merosomes in their blood, and these may be early markers of pathology. However, potential donors whose blood contains merosomes are typically deemed eligible to donate and do in fact donate because there is no readily available screening tool or method for detecting merosomes in whole blood in a typical blood donation setting. From the perspective of screening the blood of potential donors it would be clinically very helpful to have available an apparatus and method that allowed a clinician to get a sample of whole blood from the potential donor and analyze the sample spectrophotometrically for the presence of merosomes. Rapid detection of merosomes before a blood donation is made could be an effective tool for eliminating some donors from donating. Just as importantly, rapid merosome detection could be a tool for identifying that a patient's blood needs further analysis. However, a merosome test would only be practical and useful if the initial screening could be performed at point-of-care or in a doctor's office on a finger prick sample, avoiding venous puncture.

In order for a merosomes test to be useful for practical screening in the context of most blood donations it is necessary to get a sample of whole blood. The accessible sample might contain interfering particles like red blood cells (RBCs) in whole blood or in red cell concentrates. But before a spectrophotometric merosomes test can be effective it is necessary to remove RBCs because they block all light transmitted through the sample and thus interfere with the test. From a research perspective it is feasible to gently centrifuge a sample of whole blood to sediment the RBCs, leaving a supernatant that may be analyzed for merosomes (in this context the word gentle means low speed centrifugation, which in turn takes a long time). However, from a clinical blood donation perspective centrifugation is not practical. Among other reasons, centrifugation, which can take up to 12 minutes to complete, takes more time than is normally available and requires a larger sample of whole blood from the potential donor than is warranted prior to actual donation. Reducing time by increasing centrifugation speed is not an option because it would be changing the merosome concentration (any factor or particle that is produced as a function of activation would be increased). The primary risk is that the higher shear stress at higher centrifugation rate could activate platelets to fragment off merosomes and thus cause false results.

Filtration of whole blood is another possible alternative method to remove RBCs from whole blood for testing for merosomes. While filtration is potentially much faster than centrifugation, again, it is not a suitable clinical option because it is not possible to filter RBC without activating platelets, the very process that generates merosomes. This is mostly because RBC are very deformable as they are designed to squeeze through capillaries in the body that are much narrower than their cell size. Effective filter pore size would therefore have to be very small, increasing shear stress and the risk of platelet activation as well as blockage.

Thus, gentle separation techniques that separate RBCs are important to the present invention to avoid generation of merosomes. There are numerous patents that relate to microfluidic separation of blood components but to Applicant's knowledge, none of these techniques focus on the detection of merosomes and therefore do not focus on the need for low shear and minimized stress to preserve platelets, or they require significantly higher sample volume. These factors make the known microfluidic separators not useful in point-of-care situations. To illustrate the foregoing points, a few known patent publications that are directed to microfluidic blood separation are described below:

US 2002/0076354 A1: This patent publication mentions platelets but only as a component of blood with no consideration of their sensitivity and potential to activate during manipulation. The rotating bio-disc requires centrifugation for separation and includes a valve and is not aimed at obtaining platelet-rich plasma with a merosome concentration that is not changed by platelet activation during separation.

WO 2015/061497 A1: This patent publication includes lysis of red blood cells which would certainly affect merosome concentration in the plasma and has to be avoided in order to be useful in a system according to the present invention. The disclosure of this publication talks about sequestration pens and isolation regions (unswept regions) which have the purpose of reaction chambers rather that are not intended for separation of blood components. Moreover, all cells are called micro-objects and specific sequestration to sequestration pens is achieved by antibody capture. Because such efficiency of capture is dependent on the surface area, it would limit how many RBCs could be removed from whole blood.

WO 2017/006093 A1: This patent publication describes magnetic-activated, acoustophoresis and optical tweezers in addition to gravitational field for cell separation—for instance, the trajectory of a particle may be controlled by balancing the force applied by an acoustic or optical field with the gravitational force and wherein the gravitational field is used as an assist for the sorting process. But the gravitational field is said to not be required for separation and may in fact be irrelevant to the disclosed sorting techniques.

In view of the shortcomings of the known methods of analyzing blood for donation, blood from a finger prick and of the prior art, an improved apparatus method for spectrophotometrically analyzing blood and other body fluids for merosomes, especially in the context of blood donations at typical donation centers, remains highly desirable. Other locations such as mobile donor clinics and doctor's offices would also hugely benefit because they have significant time constraints and usually cannot perform a separate venipuncture to draw blood prior to donation or other draws—the specifics of both could be informed by the merosome concentration.

### SUMMARY OF THE INVENTION

The present invention relates to apparatus and method for separating components of a body fluid, especially whole blood, to prepare a sample that may be analyzed using spectrophotometric techniques such as dynamic light scattering (DLS) to assess the composition of the sample. More specifically, in a preferred embodiment whole blood is processed to remove red blood cells so that DLS may be used to analyze the separated sample to detect merosomes in the sample. A microfluidic separator according to the invention may be defined by a capillary tube having red blood cell traps incorporated therein; importantly, the microfluidic separator does not activate platelets as the whole blood flows through the separator by air replacement action (i.e., suction).

Accordingly, in accordance with one aspect of the present invention, there is provided a method for diagnosing a pathological condition in a patient based on a body fluid from the patient, the method comprising steps of: using a DLS instrument to collect DLS measurements from the body fluid; using the DLS measurements to detect a presence of merosomes in the body fluid; and diagnosing the pathological condition based on the presence of said merosomes, the presence of the detected merosomes being indicative of the existence of the pathological condition in the patient.

### BRIEF DESCRIPTION OF THE DRAWINGS

Further features and advantages of the present invention will become apparent from the following detailed description, taken in combination with the appended drawing, in which:

FIG. 1 is a schematic view of a first illustrated embodiment of a separator for spectrophotometric analysis of body fluids according to the invention.

FIG. 2 is a close up and schematic view of one red blood cell (“RBC”) trap in a separator for spectrophotometric analysis according to the invention.

FIG. 3 is top view of the RBC trap shown in FIG. 2, taken along the line 3-3 of FIG. 2.

FIG. 4 is a schematic view of a separator assembly for spectrophotometric analysis according to the invention including a suction generator and additional environmental and operational components.

FIG. 5 is a schematic view of a second illustrated embodiment of a separator for spectrophotometric analysis of body fluids according to the invention.

### DETAILED DESCRIPTION OF EMBODIMENTS

Various embodiments and aspects of the present invention will now be described, including a method and apparatus for screening blood from potential donors to assess the eligibility of the donor. More specifically, the invention is embodied in an apparatus and method for separating RBCs from a small sample of a potential donor's blood (such as would be obtained with a finger prick) to thereby remove particles from the blood that interfere with the measurand (i.e., the substance or particles to be measured) in the sample, and to thus permit quick analysis of the measurand by spectrophotometric tools such as dynamic light scattering (DLS).

The invention comprises a separator with which whole blood can be separated into a red blood cell fraction and a measurand fraction, such that the level of merosomes within the measurand fraction can be measured in situ using DLS, thereby avoiding the need for centrifuging or filtering the whole blood and avoiding activation of platelets or other cells that could fragment into merosomes during the separation process.

The present application relates generally to the Dynamic Light Scattering method and system as described in U.S. Pat. Nos. 8,323,922 and 8,835,129, both of which are also incorporated herein by this reference. However, the present invention relates to the use of a microfluidic separation apparatus that separates components prior to analysis of desired components in measurand by a dynamic light scattering system and methods to detect merosomes and/or nanoparticles in blood and other body fluids, as an indicator of the presence of disease, an indicator of a risk of disease, and/or as a means of monitoring and assessing the eligibility of the blood for donation.

The term “merosomes” as used herein is understood to mean particles within body fluids (such as blood), which have a hydrodynamic radius of less than about 1 micrometer, and may in one possible embodiment have a hydrodynamic radius of between approximately 20 and 1000 nm, and more preferably in another embodiment may have a hydrodynamic radius of between about 50 nm and 550 nm. The term merosomes as used herein is also intended to include so-called “nano-particles”. The term “merosomes” is used interchangeably with the term “microparticles.” Merosomes or microparticles are much smaller than red blood cells (RBCs) or platelets in a platelet rich plasma blood sample for example.

Although the present method of using DLS is primarily intended as a technique for detecting merosomes in a whole blood or platelet rich plasma sample as a means of assessing the eligibility of the blood for donation, it can be applied to measuring merosome levels in other body fluids, such as other blood products, urine, synovial fluid, cerebrospinal fluid, tears, as well as other biological fluids and colloids where contaminating particles need to be removed to enable DLS testing.

Merosomes are important for numerous aspects of analyzing body fluids in addition to platelet concentrates. Applicant's U.S. Pat. No. 8,323,922 describes in detail the importance of analyzing merosomes in platelet concentrates as a part of diagnosing a pathological condition. For example and as contemplated in the '922 patent, merosomes present in blood can be an early marker of pathology in a blood donor who otherwise would be considered to be an eligible donor. Thus, measuring microparticles, merosomes in the donor blood or any component (e.g., platelets, red blood cells, plasma) is considered a diagnostic measure of a pathological condition as described in the '922 patent. However, in order for a merosome test to be useful for practical screening of donor blood it is necessary to get a sample from whole blood or a red blood cell concentrate but remove the red blood cells (RBC) before the sample can be tested by dynamic light scattering techniques as described herein. The primary reason is that RBCs block all or most of the light in the DLS or any other photometric analysis and thus interfere with the test. For research purposes it is feasible to centrifuge the whole blood sample to sediment the RBC. However, as noted previously, RBC sedimentation by gentle centrifugation is not practical for routine analysis because it can activate platelets and cause generation of merosomes.

Filtration of the whole blood sample would separate the RBC. However, filtration of RBC results in activation of the platelets, primarily because RBC are very deformable as they are designed to squeeze through capillaries in the body that are much narrower than their cell size. And microfluidic devices have been used to separate RBC from the rest of the blood but as far as Applicant is aware, this has been done in order to test the RBCs. It is therefore a primary object of the present invention to describe apparatus and methods for separating RBCs from whole blood (or other body fluids) without activating platelets so that the resultant fluid may be analyzed using DLS procedures to detect merosomes to thereby screen donors.

The separator according to the present invention as described herein and shown in the drawings is needed to remove particles that interfere with the measurand (the substance or particles to be measured) in the sample. The separator allows very gentle removal of the interference in order to avoid changing the concentration of the measurand. This is always the case when the measurand is an indicator of cell activation and standard separation techniques such as centrifugation or filtration are a source of stress on the cells to be separated from the measurand, or to be separated with the measurand from other particles, so that the process of preparing the sample for testing changes the component to be tested and false results are obtained.

For example, blood cells, especially platelets bud off small fragments when exposed to stress such as shear stress, low or high temperatures, stimulating chemicals, irradiation or any non-physiologic condition. These fragments could be exosomes, microparticles, extracellular vesicles or more generally merosomes, i.e., parts of the original cell. The concentration of merosomes circulating in the blood of a person could be an indicator of a pathological condition such as an autoimmune disease. However, at least in the context of DLS analysis of RBCs and flow cytometry, measuring the concentration of merosomes would require isolation of these small particles because both the cells that formed them and other cells are interfering with their detection. DLS is capable of measuring microparticles in samples containing platelets and other cells, which are present in relatively low concentration.

In the case of platelet merosomes separation from red blood cells is currently performed by either centrifugation or filtration, both of which have the potential to activate platelets and cause further formation of merosomes and affect the result of the measurement.

In order to reduce the time, shear stress and temperature stress of separation for small sample volumes, the current invention is using a flow separator that traps large particles such as red blood cells—but not limited to red blood cells—while the sample is drawn into a container such as a capillary and is then immediately testable for the measurand.

Another example would be free hemoglobin from red blood cells which needs to be separated from the cell-contained hemoglobin for example when the extent of hemolysis needs to be determined. State of the art technology is to centrifuge the sample prior to free hemoglobin testing; however this centrifugation step can cause lysis of red blood cells, increase the amount of free hemoglobin and thus affect the result.

Microfludic apparatus for blood cell separation is generally known in the prior art. While the end result of the current invention and the prior art is similar—separation of large particles from a supernatant—one substantial difference in this invention is that the large particles are not the measurand and are viewed as interferences themselves and upon exposure to stress will negatively affect the measurand in the supernatant. Therefore, the separation in the current invention has to fulfill very specific requirements to ensure separation without cell activation and avoid falsification of the results. Further this invention does not aim to concentrate any particles but instead aims to avoid any change in concentration of merosomes or molecules in the sample so that the result could have diagnostic value.

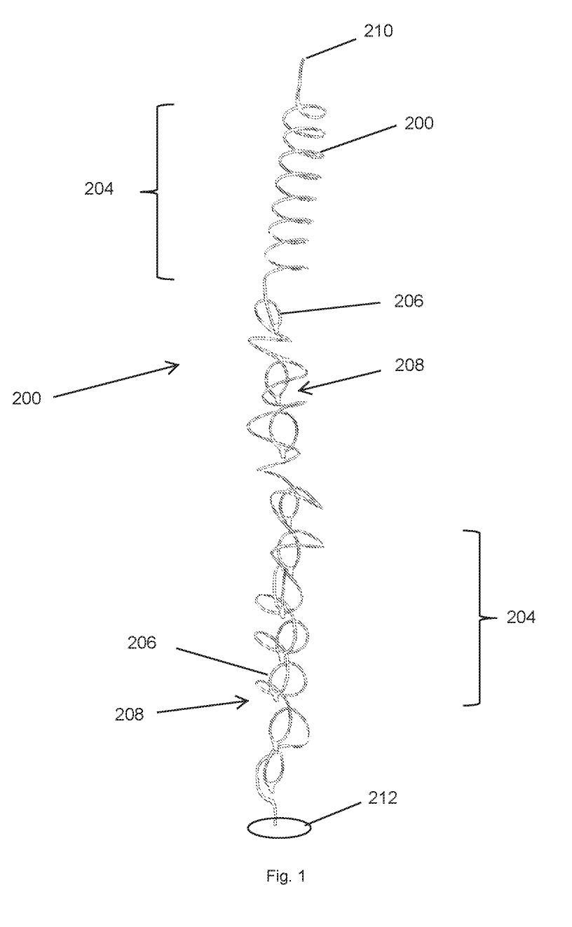

With reference now to FIG. 1, a microfluidic separator 200 according to an embodiment of the invention is shown. Generally described, the microfluidic separator 200 is a capillary tube 202 through which whole blood may be drawn upwardly through the tube in the direction of arrow A (for example, from a drop of whole blood 212 obtained from a finger puncture) by suction or capillary action. Preferably, the whole blood 212 is processed with anticoagulation or whole blood fixation techniques prior to separation using the separator 200 described herein. Fixation of the whole blood avoids clotting and platelet activation; it also, as noted below, requires that the materials used for the separator are aldehyde resistant.

Separator 200 is defined by a microfluidic tube 202 that defines a fluid pathway and which is preferably formed of an aldehyde resistant material that is formed to include plural spiral sections such as shown with reference number 204. Some of the spiral sections 204 define downward loop portions 206 that are positioned physically below the adjacent portions of the capillary tube 202 on either side of the downward loop portions. Accordingly, the downward loop portions 206 of the capillary tube 202 define red blood cell traps 208. Because the RBCs are the heaviest component of whole blood, as the whole blood flows upwardly in the capillary tube 202 (arrow A) as the tube is held generally vertically, the RBCs move the slowest relative to other components of the blood and the RBCs are retained in the RBC traps 208 under the force of gravity. Blood is drawn upwardly in tube 202 via flow induction caused by capillary action or with gentle suction applied to the tube. As such, the components in the whole blood are not agitated such as would occur with more aggressive separation methods such as hydrodynamic separation. RBCs are, for example, not ruptured and platelets are not activated. It will be appreciated that the structure of separator 200 shown in the drawings is illustrative and there are other structures that are functionally equivalent. As just one example, the downwardly extending loop portions 206 could be formed wider and with less depth. Other alternatives that accomplish the same functionality will be apparent from the disclosure herein.

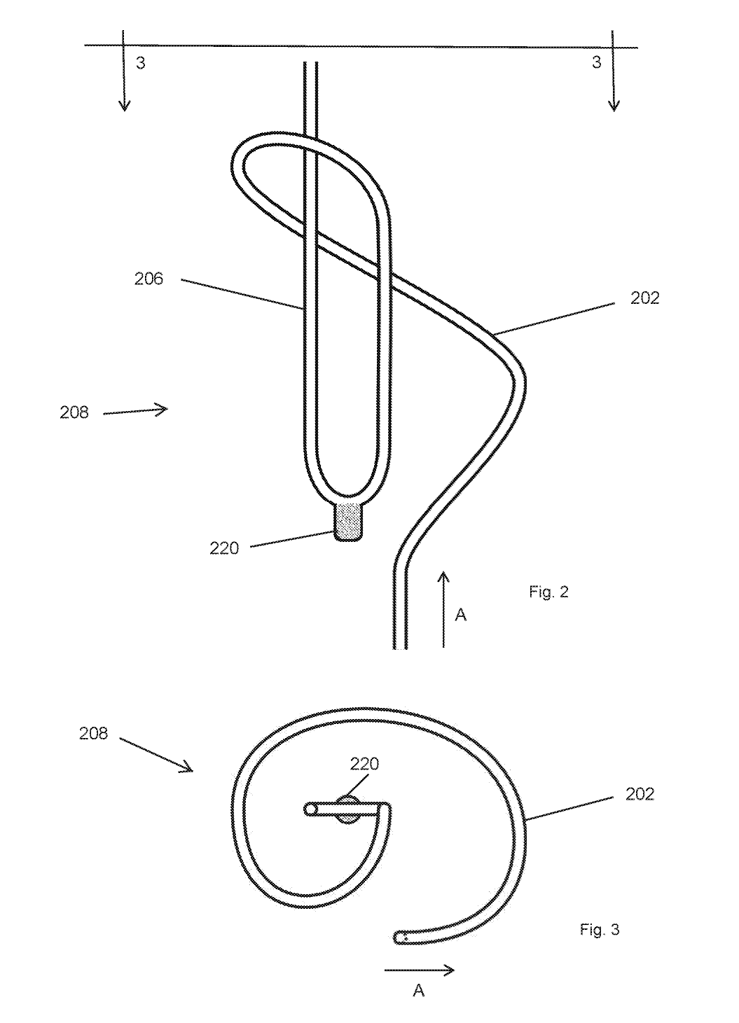

A close up of a single RBC trap 208 is shown in FIG. 2 in side elevation view and in FIG. 3 in a top plan view. It may be seen that the capillary tube 202 has a downwardly extending loop portion 206 that terminates at a cell trap 220 at the lowermost extent of the loop portion. RBCs are retained in the cell trap 220 by the action of gravitational force as blood flows upwardly in the tube 202 as shown by the arrows. The trap 220 could have various orientations and shapes.

The sample that is obtained from the top 210 of the capillary tube 202 is either an RBC supernatant or a platelet rich plasma (PRP) that may be beneficially analyzed by the DLS techniques described herein. While the resulting sample may include white blood cells, the presence of those cells does not interfere with the DLS analysis of the sample to detect merosomes.

The invention described herein and as shown in the drawing does not utilize hydrodynamic separation techniques (such as centrifugation and other cyclonic separators) because hydrodynamic separation requires sheath fluid (i.e., carrier liquid=auxiliary fluid=sheath fluid=fluid for hydrodynamic focusing), which would not be acceptable for purposes herein. Instead, the invention provides for a very small volume of sample (such as a drop of blood) to have components separated very gently and very quickly with no external pumps or power source (other than suction from a pipette or bulb, or flow induction resulting from capillary motion). There is no membrane penetration device such as a needle. RBC separation is achieved with RBC traps that accumulate RBCs by gravitational separation alone. It will be appreciated that as used herein the terms gravity and gravitational force refer to the vertical direction that is normal to a horizontal ground plane—i.e., a force vector in the vertically downward direction.

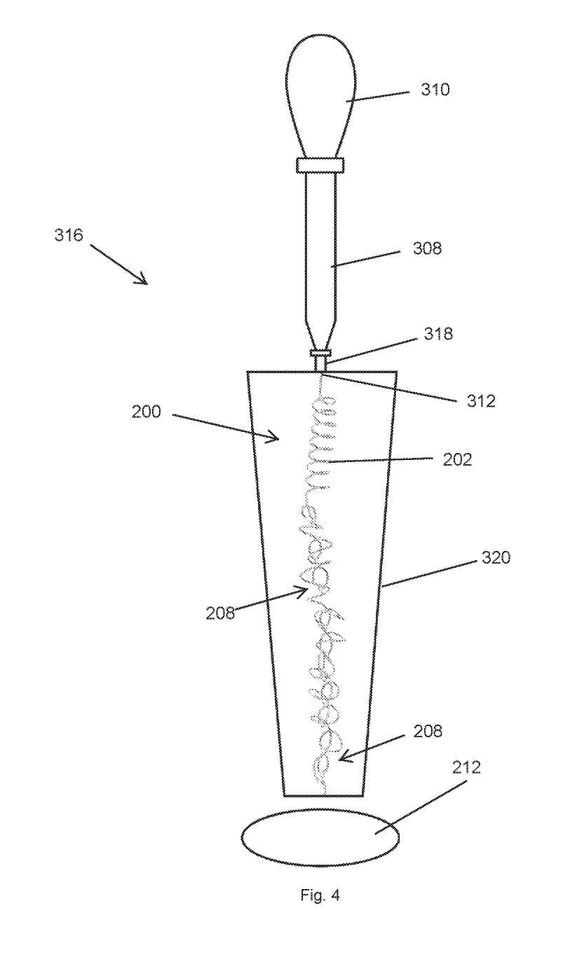

In practice, and with reference to FIG. 4, the separator 200 is ideally adapted to be used in a setting such as a blood donation center. In FIG. 4 the separator 200 is shown as an assembly 316 with components including a pipette 308 having a suction-generating bulb 310 and an enclosure 320 in which the separator 200 is retained—the enclosure 320 makes the assembly 316 easier for clinicians to handle and manipulate in practice. A potential donor is finger punctured to obtain a drop of blood. Anticoagulation is necessary so that the blood does not coagulate and thereby create additional, undesired merosomes. A conventional anticoagulant such as heparin or EDTA may be provided as a powder or coating, for example on or coating the interior surface of the inlet opening of the spiral sections 204 to the capillary tube 202 or otherwise. And as noted above, whole blood fixation may be utilized prior to separation with the separator 200. The tip, inlet opening 304 is brought into contact with the drop of blood 212 and suction, as for example with a bulb 310 fitted to pipette 308 is applied to the top 312 of the capillary tube 202 (the top 312 of tube 202 is attached to pipette 308 with an appropriate fitting such as a conventional Luer Lock 318). As whole blood is drawn upwardly through the tube 202 the heavier RBCs are trapped in the traps 206 and the product that is extracted from the top is either an RBC supernatant or a platelet rich plasma (PRP) that may be beneficially analyzed by the DLS techniques described herein. As noted above, while the resulting sample may include white blood cells, the presence of those cells does not interfere with the DLS analysis of the sample to detect merosomes. DLS analysis is used to make an assessment of the donor's eligibility for donation. It will be appreciated by those of skill in the art that the drawing of FIG. 4 is highly schematic. Among other things, the pipette 308 shown in the drawing could be of optical quality and could directly be the container used as the sample-containing vessel in the DLS instrument.

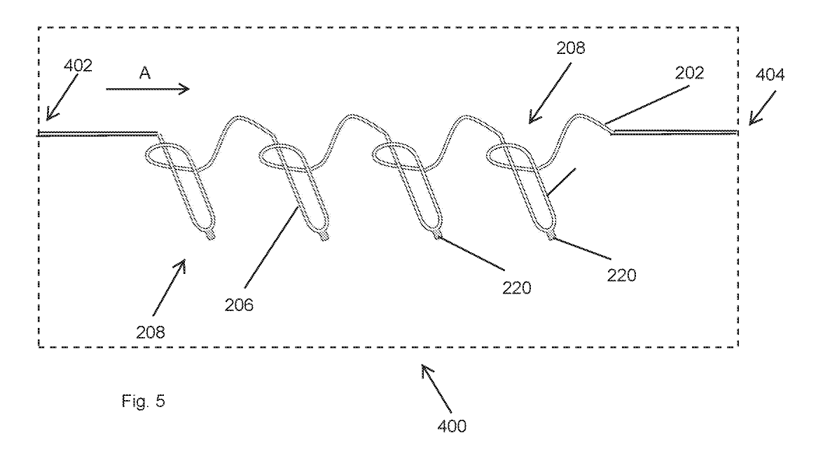

It will further be appreciated that the separator 200 shown in the drawings and described herein may be embodied in a microfluidic chip that contains the capillary tube 202 formed in the interior of the chip, which may include plural capillary tubes as part of the chip. As described above, fixation of whole blood is preferred prior to separation and as such it is necessary that the capillary tube 202 in the chip be aldehyde resistant. One example of a microfluidic chip that embodies a separator 200 according to the invention is shown schematically in FIG. 5, and described below. It will further be appreciated that the force that induces a flow of blood into the capillary tube may be with a suction-inducing device such as that described above, or a flow may be induced by capillary action, or motion, alone.

It should be emphasized that even where a sample is fixed prior to analysis with the invention described herein the invention is the preferred way of separating even a fixed sample because the inventive apparatus and method are fast and the crosslinking of proteins on the cell surface or of plasma proteins is expected to clog filters faster and change electric and possibly acoustic properties of RBC while the gravitational separation described herein is unaffected. In other words, the same separator as described in herein may be used with or without fixation while separators based on other principles might have to be modified where the sample that is being separated has been fixed.

With reference now to FIG. 5, it may be seen that the capillary tube 202 is embedded in a microfluidic chip 400 (shown schematically in phantom lines) and may be oriented in a generally horizontal array in which the spiral sections 204 define downward loop portions 206 that are positioned physically below the adjacent portions of the capillary tube 202 on either side of the downward loop portions. Capillary tube 202 includes an inlet 402 and an outlet 404. As with the embodiment of FIG. 1, in the embodiment of FIG. 5 the downward loop portions 206 of the capillary tube 202 define red blood cell traps 208. To reiterate, because the RBCs are the heaviest component of whole blood, as the whole blood flows through the generally horizontal capillary tube 202 (arrow A) the RBCs move the slowest relative to other components of the blood and the RBCs are retained in the RBC traps 208 under the gravitational force.

While the present invention has been described in terms of preferred and illustrated embodiments, it will be appreciated by those of ordinary skill that the spirit and scope of the invention is not limited to those embodiments, but extend to the various modifications and equivalents as defined in the appended claims.

1. Apparatus for separating components contained in body fluid to generate a measurand for spectrophotometric analysis, comprising:
a tube defining a fluid pathway having an inlet and an outlet and at least one trap, the at least one trap defined by a loop portion of the tube that is positioned beneath adjacent portions of the tube on either side of the loop portion.

2. Apparatus according to claim 1 including a flow inducer for inducing flow of a body fluid from a source thereof through the inlet into the fluid pathway and out of the outlet.

3. Apparatus according to claim 2 wherein the flow inducer comprises a suction generator connected to the outlet.

4. Apparatus according to claim 2 wherein tube is a capillary tube and the flow inducer comprises capillary action.

5. Apparatus according to claim 1 in which the tube comprises aldehyde resistant material.

6. Apparatus according to claim 1 in which the body fluid is blood.

7. Apparatus according to claim 6 in which the measurand comprises merosomes.

8. Apparatus according to claim 7 including an anticoagulating agent in the fluid pathway.

9. Apparatus according to claim 8 in which red blood cells are retained in the least one trap under the force of gravity alone as blood flows from the inlet through fluid pathway and the trap and toward the outlet.

10. Apparatus according to claim 1 in which the spectrophotometric analysis comprises dynamic light scattering analysis.

11. A method of separating components contained in body fluid to generate a measurand for spectrophotometric analysis, comprising the steps of:
a. drawing body fluid into a tube inlet;
b. passing the body fluid through a trap formed in the tube, wherein the trap is defined by a loop portion of the tube that is positioned beneath adjacent portions of the tube on either side of the loop portion to thereby retain some components in the trap under the force of gravity to thereby generate a measurand;
c. withdrawing the measurand from the tube at a tube outlet; and
d. spectrophotometrically analyzing the measurand.

12. The method according to claim 11 wherein the measurand comprises merosomes.

13. The method according to claim 10 in which the step of drawing body fluid into the tube comprises applying suction to the tube outlet.

14. The method according to claim 10 in which the step of drawing body fluid into the tube comprises capillary action.

15. The method according to claim 11 in which the body fluid comprises whole blood and wherein red blood cells are the components retained in the trap.

16. The method according to claim 15 including the step of exposing the whole blood to an anticoagulating agent in the tube and wherein platelets in the whole blood are not ruptured as they flow through the flowpath.

17. The method according to claim 11 in which the components retained in the trap comprise red blood cells.

18. The method according to claim 11 including the step of fixation of the whole blood prior to the whole blood is drawn into the tube.

19. Apparatus for separating components contained in body fluid to generate a measurand for spectrophotometric analysis, comprising:
a tube defining a fluid pathway having an inlet and an outlet and plural traps between the inlet and the outlet, wherein each trap is defined by a loop portion of the tube that is positioned beneath adjacent portions of the tube on either side of the loop portion;
a flow inducer for inducing a flow of body fluid through the tube; and
an anticoagulant in the fluid pathway.

20. Apparatus according to claim 19 including an anticoagulant in the fluid pathway.
